(19) United States
(12) Patent Application Publication
(10) Pub. No.: US 2024/0217035 A1
(43) Pub. Date: Jul. 4, 2024

(54) FILMS AND METHODS FOR CUTTING OR PERFORATING A WEB WITH A LASER

(71) Applicant: Kimberly-Clark Worldwide, Inc., Neenah, WI (US)

(72) Inventors: Peiguang Zhou, Suwanee, GA (US); Wade R. Thompson, Cumming, GA (US); Vikram Kaul, Atlanta, GA (US)

(21) Appl. No.: 18/563,457

(22) PCT Filed: May 26, 2021
(86) PCT No.: PCT/US21/34140

§ 371 Date: Nov. 22, 2023

(51) Int. Cl. B23K 26/38; B23K 26/402; B23K 103/00; B23K 103/16; C08K 3/26; C08K 3/30

(52) U.S. Cl. CPC B23K 26/38; B23K 26/402; C08K 3/26; C08K 3/30; B23K 2103/16; B23K 2103/42; C08K 2003/265; C08K 2003/3045

(57) ABSTRACT

Films and methods for cutting or perforating webs including films are disclosed. The films can include a polymer and a plurality of particles. The plurality of particles can include absorbent filler particles and void generating filler particles. The absorbent filler particles can provide between about 1% to about 40% by weight of the film. The void generating filler particles can provide between about 10% to about 40% by weight of the film. The absorbent filler particles can be a different particle than the void generating particles. The film can also include a plurality of voids.

### TECHNICAL FIELD

The present disclosure relates to films and methods for cutting or perforating a web with a laser. More specifically, the present disclosure relates to films and methods for cutting or perforating a web including a film with particles with a laser.

### BACKGROUND OF THE DISCLOSURE

Laser cutting technology provides a non-contact, flexible cutting system that is used in a variety of manufacturing applications for cutting or perforating a web. An exemplary application for laser cutting is in cutting at least a portion of an absorbent article chassis, such as a leg cutout, in a web of interconnected absorbent articles. The laser cutting system provides the ability to modify the leg cutout shape electronically when a manufacturing line changes between absorbent articles of different step sizes, grades, or between different absorbent articles altogether. An exemplary laser that can be used in such cutting can be a CO₂ laser, for example, having a wavelength of 980 cm⁻¹.

Polyolefin-based films are common to absorbent articles in their use as liquid impermeable barrier, and often need to be cut for a preferred shape in the leg opening of the absorbent article. However, laser cutting the polyolefin based film at high speeds provides difficulties, such that the speed of the machine at such a module may be limited by the ability of the laser assembly to achieve a satisfactory cut. This is especially true for polyethylene films, such as low density polyethylene (LDPE) or linear low density polyethylene (LLDPE).

Accordingly, there is a desire for improved methods and systems for cutting or perforating a film, or a web including a film, with a laser that allows for increased cutting speeds and improved cut edge characteristics. There is also a desire for a film, or a web including a film, that can be cut or perforated at such higher speeds and/or that provide an improved cut edge.

### SUMMARY OF THE DISCLOSURE

In one embodiment, a film can include a polymer and a plurality of particles. The plurality of particles can include absorbent filler particles that provide about 1% to about 40% by weight of the film. The plurality of particles can also include void generating filler particles that provide between about 10% to about 40% by weight of the film. The absorbent filler particles can be a different particle than the void generating particles. The film can also include a plurality of voids.

In another embodiment, a method for cutting or perforating a web can include providing a web including a film. The film can include a polymer and a plurality of particles. The plurality of particles can include absorbent filler particles that provide about 1% to about 40% by weight of the film. The plurality of particles can also include void generating filler particles that provide between about 10% to about 40% by weight of the film. The absorbent filler particles can be a different particle than the void generating particles. The method can also include stretching the film to generate a plurality of voids in the film. The method can additionally include providing a laser assembly. The method can include directing a beam of light from the laser assembly upon the surface of the web with relative movement between the beam of light and the web to cut or perforate the web.

### BRIEF DESCRIPTION OF DRAWINGS

A full and enabling disclosure thereof, directed to one of ordinary skill in the art, is set forth more particularly in the remainder of the specification, which makes reference to the appended figures in which:

FIG. 1 is a perspective view of a web of film being stretched.

FIG. 2 is a graph depicting the infrared (IR) spectra absorbance.

FIG. 3 is a top view of a web of interconnected absorbent assemblies, including the film of FIG. 1, and depicts an exemplary absorbent assembly being cut from the web and rotated.

FIG. 4 is a top view of an exemplary web of interconnected absorbent articles, each including an absorbent assembly such as depicted in FIG. 3, and depicts an exemplary leg cutout path completed by a laser assembly.

FIG. 5 is a graph depicting the laser cutting speed for various exemplary codes as listed in Table 5.

FIG. 6A is scanning electron microscope (SEM) image of a cross-section of an exemplary film including void generating filler particles with no stretching.

FIG. 6B is an SEM image of a cross-section of an exemplary film including void generating filler particles after a stretching at a stretch rate of 250%.

FIG. 6C is an SEM image of a cross-section of an exemplary film including void generating filler particles after a stretching at a stretch rate of 410%.

FIG. 7 is a graph depicting void percentage of exemplary films in comparison to a stretch rate of exemplary films of Table 7.

FIG. 8 is a graph depicting cutting speed in comparison to cutting angle off of the stretching direction of the film for various films of Table 8.

FIG. 9 is a graph depicting the infrared (IR) spectra absorbance for the a PE film in comparison to a PE film with an exemplary coating material.

Repeat use of reference characters in the present specification and drawings is intended to represent the same or analogous features or elements of the disclosure.

### DETAILED DESCRIPTION OF THE DISCLOSURE

In an embodiment, the present disclosure is generally directed towards films and methods for cutting or perforating a web including a film with a laser, in which the web includes a film that has a plurality of particles and that has been stretched to create a plurality of voids. These methods can increase the laser cutting speed in which the film can be cut or perforated at and can improve the overall cut quality and softness of the cut edge. Each example is provided by way of explanation and is not meant as a limitation. For example, features illustrated or described as part of one embodiment or figure can be used on another embodiment or figure to yield yet another embodiment. It is intended that the present disclosure include such modifications and variations.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. As used herein, the terminology of “first,” “second,” “third”, etc. does not designate a specified order, but is used as a means to differentiate between different occurrences when referring to various features in the present disclosure. Many modifications and variations of the present disclosure can be made without departing from the spirit and scope thereof. Therefore, the exemplary embodiments described above should not be used to limit the scope of the invention.

### Definitions

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products, including, but not limited to, menstrual pads or pants, incontinence products, medical garments, surgical pads and bandages, other personal care or health care garments, and the like without departing from the scope of the present disclosure.

The term “acquisition layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded” or “coupled” refers herein to the joining, adhering, connecting, attaching, or the like, of two elements. Two elements will be considered bonded or coupled together when they are joined, adhered, connected, attached, or the like, directly to one another or indirectly to one another, such as when each is directly bonded to intermediate elements. The bonding or coupling of one element to another can occur via continuous or intermittent bonds.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “polymers” include, but are not limited to, homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to isotactic, syndiotactic and atactic symmetries.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5 and 10 and about 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a material which softens and which can be shaped when exposed to heat and which substantially returns to a non-softened condition when cooled.

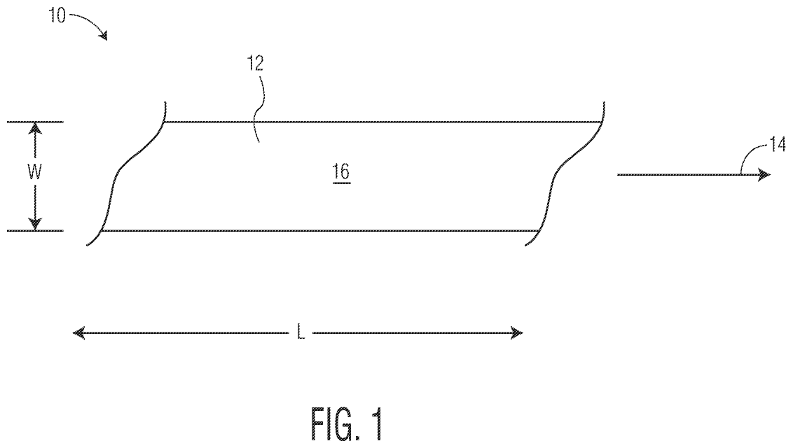

Referring to FIG. 1, a web 10 being stretched is illustrated. The web 10 can be a layer of film 12. The film 12 can be a monolayer of film, or can be a multilayer film. In the context of a multilayer film, the film 12 can include one or more skin layers and one or more tie layers between layers of the film 12. In a multilayer film 12, the various layers can have the same or different components, such as various polyolefin polymers and/or particles.

The film 12 can include a polymer. For example, the film 12 can be comprised of a polyolefin such as polyethylene, polypropylene, or combinations thereof. In one embodiment, the film 12 can be comprised of a linear low density polyethylene (LLDPE). As used herein, “linear low density polyethylene” refers to polymers of ethylene and higher alpha olefin comonomers, such as C₂-C₁₂ comonomers, and combinations thereof, having a density of about 0.900 to 0.935 grams/cm³. In another embodiment, the film 12 can be comprised of a low density polyethylene (LDPE). As used herein, “low density polyethylene” refers to a polyethylene having a density between about 0.91 and 0.925 grams/cm³. It is to be contemplated that a film 12 may have various other polymers and be still be within the scope of this disclosure.

The film 12 can additionally include a plurality of particles. The plurality of particles can include absorbent filler particles. “Absorbent filler particles,” as used herein, include particles that provide sufficient absorption of laser light/energy to cause particle heating to aid in film cutting. In preferred embodiments, absorbent filler particles include absorbance values of greater than 0.15 based on the FT-IR measurements at wavelengths between 9.0 μm and 11.0 μm. IR spectra absorbance can be acquired using a Spectra Tech Golden Gate Single bounce ATR accessory equipped with a diamond cell ATR crystal on a Nicolet Nexus 670 FTIR, averaging 32 scans per sample at 4 cm⁻¹ resolution. Specific IR Experimental Conditions included Data Collection Information of: number of scans: 32; collection length 38.5 sec; resolution: 4.000; levels of zero filling: 0; number of scan points: 8480; number of FFT points: 8192; laser frequency: 15798.3 cm⁻¹; interferogram peak position: 4096; apodization: Happ-Genzel; phase correction: Mertz; number of background scans: 64; background gain: 8.0. The spectrometer information included: spectrometer: Nexus 670; source: IR; detector: DTGS KBr; smart accessory ID: unknown; beamsplitter: KBr; sample spacing: 2.0000; digitizer bits: 20; optical velocity: 0.6329; aperture: 100.00; sample gain: 8.0; high pass filter: 200.0000; low pass filter: 11000.0000. Exemplary absorbent filler particles and their respective absorbance values based on FT-IR measurements at 10.2 μm include, but are not limited to: BaSO₄ (0.19), BaPO₄ (1.05), carbon black (0.75), and kaolin (Theile Kaoplate 13P) (0.49). A particularly preferred absorbent filler particle can include BaSO₄.

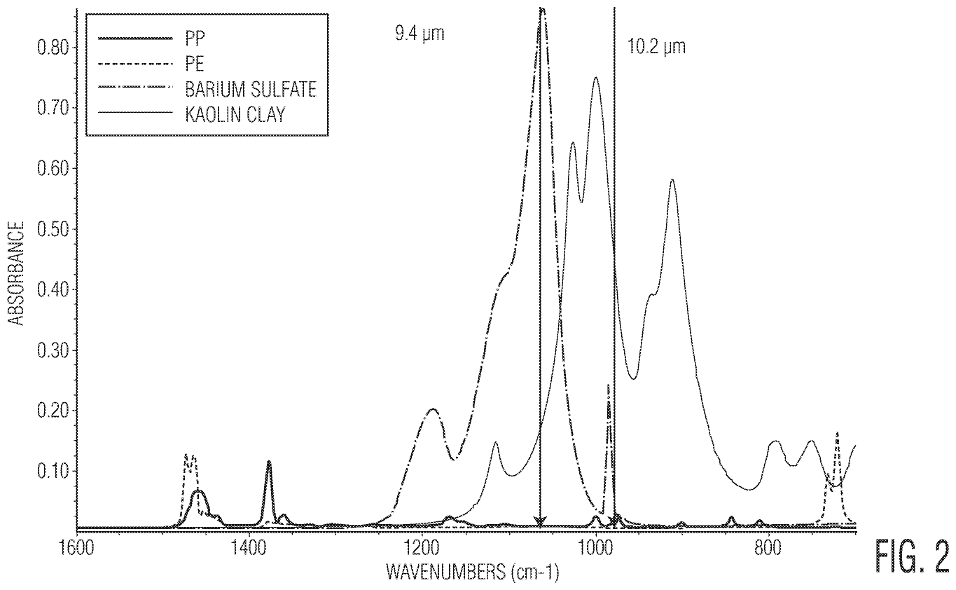

FIG. 2 illustrates the absorbance values across various IR wavelengths for materials of polypropylene (PP), polyethylene (PE), Barium Sulfate (BaSO₄), and Kaolin clay. As depicted in FIG. 2, the Barium Sulfate and the Kaolin clay have significantly higher absorbance values at 9.4 μm and 10.2 μm wavelengths, and thus, are believed to provide enhance heating to aid in film cutting.

The absorbent filler particles can be provided at various concentrations in the film 12. For example, in some embodiments, the absorbent filler particles in the film 12 can provide a concentration from about 1% to about 40% in the film 12, or more preferably from about 5% to about 30%, or even more preferably from about 10% to about 25% of the film 12 (by total weight of the film 12).

The plurality of particles in the film can additionally include void generating filler particles. “Void generating filler particles,” as used herein, include particles that can generate voids of at least about 0.2 μm to less than about 3.0 μm in a film during solid state stretching of the film of about 50% to about 500% stretch. Void sizes are measured using the ImageJ software analysis of SEM images as described further herein. Exemplary void generating filler particles can include, but are not limited to, CaCO₃, lumina, silica, clay, sand, superabsorbent particles, thermal starch, and cellulose fiber. A particularly preferred void generating filler particle can include CaCO₃.

The void generating filler particles can be provided at various concentrations in the film 12. For example, in some embodiments, the void generating particles in the film 12 can provide a concentration from about 10% to about 50% in the film 12 (by total weight of the film 12), or more preferably from about 12% to about 35% of the film, or even more preferably from about 15% to about 30% of the film 12 (by total weight of the film 12).

In some embodiments, the absorbent filler particles can be the same particles as the void generating filler particles. For example, some embodiments can include BaPO₄ or Kaolinite particles that can provide the functionality of absorbent filler particles noted above as well as the functionality of the void generating particles noted above. However, in some preferable embodiments, the film 12 can utilize absorbent filler particles that are different particles than the void generating filler particles. In other words, in some preferable embodiments, the film 12 can utilize absorbent filler particles that are a different composition than the void generating filler particles.

The absorbent filler particles and/or the void generating filler particles can be added prior to extrusion of the film 12, using techniques known by one of ordinary skill in the art. If the film 12 is a multilayer film, absorbent filler particles and/or the void generating filler particles can be added to one or more layers of the film 12. In some embodiments, the absorbent filler particles can be provided in the same layer of the film 12 as the void generating filler particles. In other embodiments, the absorbent filler particles can be provided in a different layer of the film 12 than the layer in which the void generating filler particles are provided.

The film 12 can include a length L and a width W. The film 12 can define a surface 16. The length L of the film 12 can be aligned with the machine direction in which the film 12 is produced. As shown in FIG. 1, the film 12 can be stretched in a stretch direction 14. The stretch direction 14 can be aligned with the machine direction of the film 12. In some embodiments, the film 12 can be stretched in a stretch direction 14 that is perpendicular to the machine direction, or in other words, aligned with the cross direction of the film 12. In some embodiments, the film 12 can be stretched in a stretch direction 14 that is oriented at an angle with respect to the machine direction of the film 12. In some embodiments, the film 12 can be stretched in more than one direction. For example, it is contemplated that the film 12 can be stretched in a first stretch direction that is aligned with the machine direction of the film 12 and be stretched in a second stretch direction that is aligned with the cross direction of the film 12. In some embodiments, the film 12 can be stretched at a percent stretch of between about 150% to about 600%, and more preferably from about 200% to about 500%, and even more preferably from about 200% to about 400%.

In stretching the film 12 a plurality of voids can be provided in the film 12, as will be discussed and shown in further detail below. The plurality of voids can provide a void volume percentage for the film, the methodology for calculating being described further below in relation to various SEM images. In some embodiments, the void volume percentage for the film 12 can be between about 1% to about 25%, and in some embodiments from about 1.5% to about 20%, and in some embodiments from about 2% to about 15%. After stretching the film 12, the film 12 can be relaxed. In some situations, the film 12 can then be spooled in a converting operation for use at another location. In other situations, the film 12 can be stretched and transferred directly to a machine line for further processing. In some embodiments, the film 12 can be stretched prior to being cut, as described further herein. However, it is contemplated that the film 12 could be stretched while being cut in some embodiments.

Depending on the various particles in the film and the stretching employed, the voids can be of various average size, the methodology for calculating the average size of a void being described further below. As will discussed further below, the voids can have an average size from about 0.20 μm to about 2.50 μm, or from about 0.50 μm to about 2.00 μm.

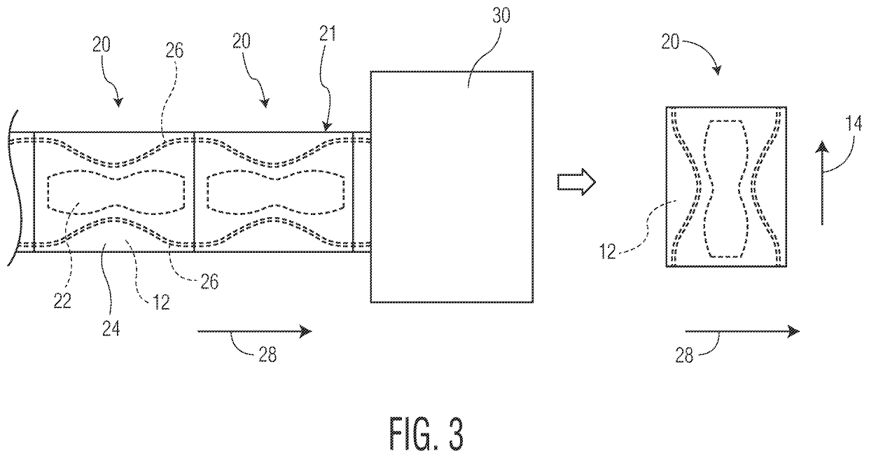

In some embodiments, the film 12 can be used to form at least a portion of an absorbent article, however, it is to be appreciated that the films 12 described herein can be utilized as a film independent of any other feature, or can be combined in various other configurations, webs, and/or products without departing from the scope of this disclosure. In one embodiment where the film 12 forms at least a portion of an absorbent article, the film 12 can form at least a portion of an absorbent assembly 20. In FIG. 3, a series of interconnected absorbent assemblies 20 are shown being transferred in the form of a web 21. An absorbent assembly 20 can include components, including, but not limited to, the film 12, an absorbent body 22 including cellulosic fibers and/or superabsorbent material, one or more non-woven layers 24, and elastic material 26. In some embodiments, the film 12 can form at least a portion of an outer cover for an absorbent article. In some embodiments, a non-woven layer 24 can form a bodyside liner for an absorbent article. If included in the absorbent assembly 20, the elastic material 26 can be in various forms, such as elastic strands as depicted. The elastic strands can help form a leg gasket on an absorbent article and provide enhanced fit. It is to be contemplated that the absorbent assemblies 20 depicted and described herein are merely exemplary, and could be provided in a variety of configurations without departing from the scope of this disclosure.

As also depicted in FIG. 3, the web 21 of absorbent assemblies 20 can be initially transferred in a direction 28 that is parallel to the stretch direction 14 of the film 12, as previously depicted in FIG. 1. In some embodiments, the web 21 of absorbent assemblies 20 can be transferred utilizing known conveying equipment (not shown for clarity) to a rotating module 30 that can cut individual absorbent assemblies 20 from the web 21 and rotate the individual absorbent assemblies 20. The general construction and operation of such a rotating module 30 is well known and is exemplified by U.S. Pat. Nos. 5,716,478 and 5,759,340 issued to Boothe et al. and U.S. Pat. No. 6,139,004 issued to Couillard et al., each of which is incorporated herein by reference in its entirety to the extent not inconsistent herewith. After such rotation, absorbent assembly 20 can be oriented such that the stretch direction 14 in the film layer 12 is no longer aligned with the direction 28 of transferring the absorbent assembly 20. In some embodiments, as depicted in FIG. 3, the film layer 12 of the absorbent assembly 20 can be oriented such that the stretch direction 14 in the film layer 12 is perpendicular to the direction 28 of transferring the absorbent assembly 20.

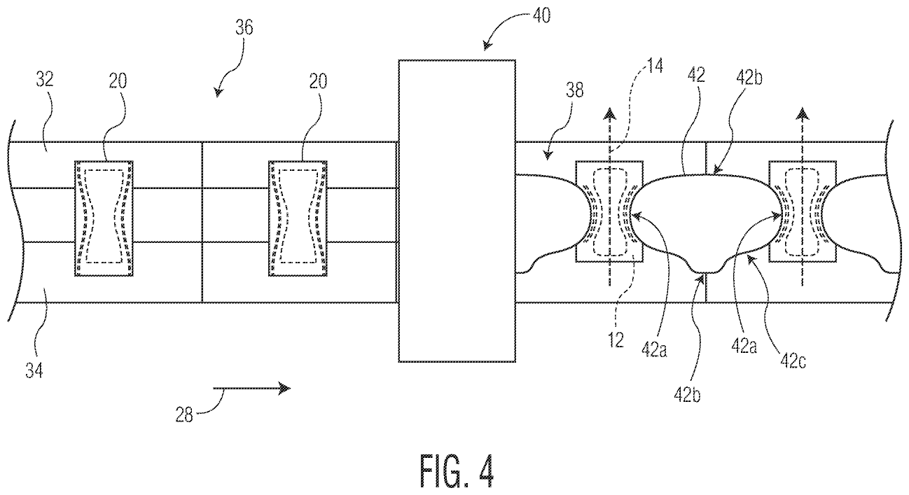

Turning to FIG. 4, in some embodiments the absorbent assembly 20 can be coupled to one or more webs after being cut and rotated by the rotating module 30. In some embodiments, the absorbent assembly 20 can be coupled to a web 32 providing front waist panel material for an absorbent article and the absorbent assembly 20 can be coupled to a web 34 providing rear waist panel for an absorbent article, each web 32, 34 including materials known to one of ordinary skill in the art such as nonwoven materials and elastic materials. In some embodiments, the absorbent assembly 20 can be coupled to such webs 32, 34 by the rotating module 30 after being cut and rotated. Coupling can occur through adhesives and/or other techniques such as pressure bonding, ultrasonic bonding, heat welding, or other suitable techniques. Once the absorbent assembly 20 is coupled to the webs 32, 34 providing front and rear waist panel material, respectively, the overall assembly provides a continuous web 36 of absorbent articles.

The web 36 of absorbent articles can then be transferred to a laser assembly 40 by transferring the web 36 in a direction 28. Alternatively, the laser assembly 40 could be moved with respect to the web 36 as well. In either case, the laser assembly can direct a beam of light from the laser assembly upon the surface of the web 36 to either cut or perforate the web 36 in at least one location by having relative movement between the beam of light and the web 36 to cut or perforate the web 36 in at least one location. As depicted in FIG. 4, the laser assembly 40 can direct the laser(s) to a surface of the web 36, such as the top surface 38 of the web 36 to cut or perforate the web 36 in at least one location. In the preferred embodiment shown in FIG. 4, the web 36 of absorbent articles is cut in a generally oval-shaped path 42 that provides leg cutouts on adjacent absorbent articles. Of course, this specific shape of the path 42 can be modified for various different absorbent articles or other products in which the methodology may be utilized without departing from the scope of this disclosure. The path 42 provided by the cut in the web 36 of absorbent articles can cut absorbent assembly 20 including the film 12. In some embodiments, such as depicted in FIG. 4, the path 42 can cut the web 32 providing the front panel material and/or the web 34 providing the rear panel material.

In some embodiments, at least a first portion 42a of the path 42 in which the laser cuts or perforates the web 36 is substantially parallel to the stretch direction 14 of the film layer 12. The first portion 42a of the path 42 in which the laser cuts or perforates the web 36 that is substantially parallel to the stretch direction 14 of the film layer 12 can be viewed near the left and right sides of the path 42 as depicted in FIG. 4. In some embodiments, another portion 42b of the path 42 in which the laser cuts or perforates the web 36 can be substantially perpendicular to the stretch direction 14 of the film layer 12. For example, this portion 42b of the path 42 that is substantially perpendicular to the stretch direction 14 can be viewed near the top and bottom of the path 42 as shown in FIG. 4. Other portions, such as portion 42c, of the path 42 can be neither parallel nor perpendicular to the stretch direction 14 of the film layer 12. Thus, one benefit to the method as described herein is the ability to cut the film layer 12 in various directions with respect to the stretch direction 14 of the film layer 12, including cutting in a direction parallel to the stretch direction 14 of the film layer 12.

One exemplary embodiment of a laser assembly 40 can be a Rofin OEM-65iX 10.25 μm 650 W CO₂ laser assembly having a focused spot size of about 210 μm in diameter (manufactured by Rofin-Sinar UK Ltd.). For experimental cutting described herein, the laser assembly 40 was set to a power of 110 W, with a pulse frequency of about 30 Khz. In some embodiments, the laser assembly 40 may have two or more lasers. However, it can be appreciated that the laser(s) from the laser assembly 40 can be operated at various wavelengths, ranging from about 9.4 μm to about 10.6 μm, or more preferably from 10.0 μm to about 10.3 μm, or even more preferably from about 10.2 μm to about 10.3 μm. The laser(s) from the laser assembly 40 can be operated at various power settings, ranging from about 65 W to about 1200 W, or more preferably from about 100 W to about 1000 W.

For purposes of testing, various film layers 12 were created with different particles and stretch rates as described in Table 1. All the cutting was completed using the laser assembly described above, with a duty cycle of 9.5%, with variance to the wavelength of the laser at a wavelength of either 9.4 μm or 10.2 μm. The maximum process cut speed listed in Table 1 was the maximum speed that was achieved that was able to provide a clean cut in the respective exemplary film layer 12.

TABLE 1
Sample Code Listing No. 1-5 and
Cut Speeds for Experimental Films

| | | | Cutting Speed 100 W (in/s) | |
|---|---|---|---|---|
| | | Stretch | 9.4 um | 10.2 um |
| Code | Description | Rate | Laser | Laser |
| LLDPE | PE film (20 gsm) | 0% | 50 | 25 |
| Nonwoven | PP Spunbond (14 gsm) | 0% | 100 | 300-400 |
| 1 | PE 64% BaSO₄ | 0% | 700 | 150-200 |
| | 36% | | | |
| 2 | 28% K-47, 72% PE | 350% | 650 | 600 |
| 3 | 28% K-13, 72% PE | 350% | 880 | >770 |
| 4A | 20% Kaolinite Op-16, | 350% | 880 | — |
| | 80% PE | | | |
| 4B | 28% Kaolinite Op-16, | 400% | — | >700 |
| | 72% PE | | | |
| 5 | 30% CaCO₃, | 0% | 150 | 125 |
| | 70% PE | | | |

As documented in the data from Table 1, codes including particles such as barium sulfate (BaSO4) and kaolinite clay (K-47, K-13, Op-16) were able to provide absorbent filler particles that absorbed heat from the IR laser and improved cutting speeds (Code Nos. 1-4B). Code No. 5 including calcium carbonate (CaCO₃) was not able to provide as high of cutting speeds as calcium carbonate does not perform as an absorbent filler particle as defined herein, but can provide some enhanced cutting speed as compared to the control code PE film.

Further exemplary codes were created were created and their compositions are described in Table 2. All the cutting was completed using the laser assembly described above, with a duty cycle of 9.5%, at 100 W and a wavelength of 10.2 μm.

TABLE 2
Additional Code Listing Nos. 6-16 and
Cut Speeds for Experimental Films

| | | | Cutting |
|---|---|---|---|
| Code | | Stretch | Speed |
| No. | Sample Description | Rate | (in/s) |
| 6 | C-85 PE 90%, BaSO₄ 10% | 0% | 150 |
| 7 | C-86 PE 80%, BaSO₄ 20% | 0% | 175 |
| 8 | C-87 PE 70%, BaSO₄ 30% | 0% | 225 |
| 9 | C-88 PE 60%, BaSO₄ 40% | 0% | 300 |
| 10 | C-89 PE 50%, BaSO₄ 50% | 0% | 350 |
| 11 | C-49 PE Dow 2047 90%, CaCO₃ 10% | 0% | 75 |
| 12 | C-50 PE Dow 2047 80%, CaCO₃ 20% | 0% | 75 |
| 13 | C-51 PE Dow 2047 70%, CaCO₃ 30% | 0% | 125-150 |
| 14 | C-52 PE Dow 2047 60%, CaCO₃ 40% | 0% | 125-150 |
| 15 | C-53 PE Dow 2047 50%, CaCO₃ 50% | 0% | 125-175 |
| 16 | C-54 PE Dow 2047 50%, CaCO₃ 50% | 350% | 250-275 |

As documented in Table 2, increasing the amount of absorbent filler particles (such as Barium Sulfate) provides for greater cutting speeds, as evidenced by code nos. 6-10. Barium sulfate provides good IR light absorption, and thus, increasing the amount of such absorbent filler particles demonstrated increased cutting speeds. Code nos. 11-13 demonstrate that increasing the amount of a poorly absorbing particle, such as calcium carbonate, does not provide much, if any, enhancements in cutting speed for a film 12. However, stretching a film 12 with calcium carbonate, which serves as a void generating filler particle, was evidenced to significantly enhance the cutting speed as shown by the comparison of code no. 16 (50% CaCO₃ with 350% stretch) that had a cutting speed of 250-275 in/sec compared to code no. 15 (50% CaCO₃ with 0% stretch) that only had a cutting speed of 125-175 in/sec.

Further experimental films 12 were created involving kaolinite (K-47) as an absorbent filler particle, and some film codes also utilized the kaolinite with the void generating filler particle of calcium carbonate (CaCO₃). Table 3 provides the results of such codes, with all the cutting being completed using the laser assembly described above, with a duty cycle of 9.5%, at 100 W and a wavelength of 10.2 μm.

TABLE 3
Additional Code Listing Nos. 17-20 and
Cut Speeds for Experimental Films

| | | | Cutting |
|---|---|---|---|
| Code | | Stretch | Speed |
| No. | Sample Description | Rate | (in/s) |
| 17 | K-47 28%, PE 72% (21 gsm) | 0% | 375 |
| 18 | K-47 28%, PE 72% (42 gsm stretched | 320% | 600 |
| | to 20 gsm) | | |
| 19 | K-47 12%, CaCO₃ | 0% | 200 |
| | 22.5%, PE 66.5% (22 gsm) | | |
| 20 | K-47 12%, CaCO₃ | 380% | 700 |
| | 22.5%, PE 66.5% (46 gsm | | |
| | stretched to 21 gsm) | | |

The results of the experimental films 12 in Table 3 demonstrate that increased cutting speeds can still be achieved when replacing a higher absorbing absorbent filler particle of Barium Sulfate with Kaolinite (K-47), when such absorbent filler particle is also combined with a void generating filler particle such as calcium carbonate (CaCO₃) and the film 12 is stretched. For example, the film 12 of code no. 20 made with 12% Kaolinite-47 and 22.5% CaCO₃ had a cutting speed of 700 in/s as compared to code no. 18 having only 28% Kaolinite-47 that demonstrated a cutting speed of only 600 in/s. Furthermore, when comparing the unstretched film 12 of code no. 19 with 12% Kaolinite-47 and 22.5% CaCO₃ the cutting speed was only 200 in/s using 100 W, thus showing the importance of stretching the film 12 to provide the void generating filler particles to create voids in the film 12 to enhance cutting speeds.

Similar results were observed with modification of the laser cutting assembly to have a wavelength of 9.4 μm as shown in the experimental films of Table 4.

TABLE 4
Additional Code Listing Nos. 21-28 and
Cut Speeds for Experimental Films

| | | | Cutting |
|---|---|---|---|
| Code | | Stretch | Speed |
| No. | Sample Description | Rate | (in/s) |
| 21 | BaSO₄ 21%, PE 79%, 20 gsm | 0% | 250 |
| 22 | BaSO₄ 35%, PE 65% (20 gsm) | 0% | 600 |
| 23 | PE 63.5%, BaSO₄ 14%, | 0% | 475 |
| | CaCO₃ 22.5% (20 gsm) | | |
| 24 | PE 63.5%, BaSO₄ 14%, | 550% | 1050 |
| | CaCO₃ 22.5% (24 gsm) | | |
| 25 | PE 56.5%, BaSO₄ 21%, | 550% | 1480 |
| | CaCO₃ 22.5% (24 gsm) | | |
| 26 | PE 56.5%, BaSO₄ 21%, | 0% | 500-525 |
| | CaCO₃ 22.5% (20 gsm) | | |
| 27 | PE 56%, BaSO₄ 14%, | 0% | 475 |
| | CaCO₃ 30% (20 gsm) | | |
| 28 | PE 56%, BaSO₄ 14%, | 550% | 1330 |
| | CaCO₃ 30% (24gsm) | | |

Table 4 demonstrates that the cutting speed of PE film can be significantly increased by combining a non-absorbent, but void generating filler particle, such as CaCO₃, with an absorbent filler particle, such as BaSO₄, and through solid state stretching with either a 9.4 or 10.2 μm wavelength laser. The highest cutting speed obtained was almost 1500 in/s with 100 W in Code 25 that utilized only 21% BaSO₄, 22.5% CaCO₃, and stretching to 550%. The importance of the void generating filler particles and stretching is shown in comparing code 25 to code nos. 21 and 22, which include 21% BaSO₄ and 35% BaSO₄, respectively, but no void generating filler particle and no stretching and only provided cutting speeds of 250 in/s and 600 in/s, respectively.

As also established in Table 4, the void generating filler particle, such as CaCO₃, does not alone improve laser cutting speeds. This is attributed to the poor absorbance properties of the laser light of this particle as discussed above and as illustrated in FIG. 2. However, incorporating a void generating filler particle, such as CaCO₃, with various higher absorbing filler particles and solid state stretching, the laser cutting speed is dramatically improved. Without being bound by theory, it is believed that the increase in laser cutting speeds is associated with the void formation from added void generating filler particles and through solid state stretching. The controllable void volume and size can be achieved by incorporating a void generating filler such as CaCO₃ and through performing preferable stretching conditions. The voids in the film promote laser cutting by amplifying laser scattering and provide a significant improvement in laser cutting speed.

Further experimental films 12 were created for comparison of the importance of various filler particles and stretching and scanning electron microscope (SEM) images were taken to analyze void size and void volume percentage. As documented in Table 5, two control codes with no particles (codes PE and PE-Stretch) were compared with codes having a void generating filler particle (CaCO₃) (code nos. 29-31) and with codes having void generating filler particles (CaCO₃) and absorbent filler particles (BaSO₄), as well as variation in stretch ratios. All the cutting was completed using the laser assembly described above, with a duty cycle of 9.5%. The thickness measurements were taken as gauge thickness, not taken from any SEM images later produced. The maximum process cut speed listed in Table 1 was the maximum speed that was achieved that was able to provide a clean cut in the respective exemplary film layer 12. Calculating the void volume and average void size was completed by image analysis using ImageJ, an open source software that is Java-based and developed by the National Institutes of Health. Cross-sectional images from each sample were binarized such that film 12 material was white in the image and space created by the voids 50 were black. The void volume percentage was calculated based on the ratio of black pixel to total pixels in the image. The average void size was calculated by inputting an ellipse into each void and taking the average of the major and minor axis of the ellipse.

TABLE 5
Additional Code Listing for Codes 29-33

| | | | | Avg. | Maximum | |
|---|---|---|---|---|---|---|
| | | | | Void | Process | Thick- |
| Code | | Stretch | Void | Size | Cut Speed | ness |
| No. | Material | Ratio | Volume | (μm) | (in/sec) | (μm) |
| PE | 100% PE | 0% | 0.01 | 0.16 | 25 | 19 |
| PE- | 100% PE | 400% | 0.10 | 0.14 | 50 | 23 |
| Stretch | | | | | | |
| 29 | 50% PE, 50% | 0% | 2.0 | 0.5 | 125 | 15 |
| | CaCO₃ | | | | | |
| 30 | 50% PE, 50% | 300% | 14.0 | 1.5 | 250 | 26 |
| | CaCO₃ | | | | | |
| 31 | 50% PE, 50% | 500% | 18.0 | 1.4 | 275 | 16 |
| | CaCO₃ | | | | | |
| 32 | 60% PE, 30% | 0% | 2.0 | 0.46 | 400 | 14 |
| | BaSO₄, 10% | | | | | |
| | BaPO₄ | | | | | |
| 33 | 60% PE, 30% | 350% | 8.0 | 0.88 | 550 | 21 |
| | BaSO₄, 10% | | | | | |
| | BaPO₄ | | | | | |

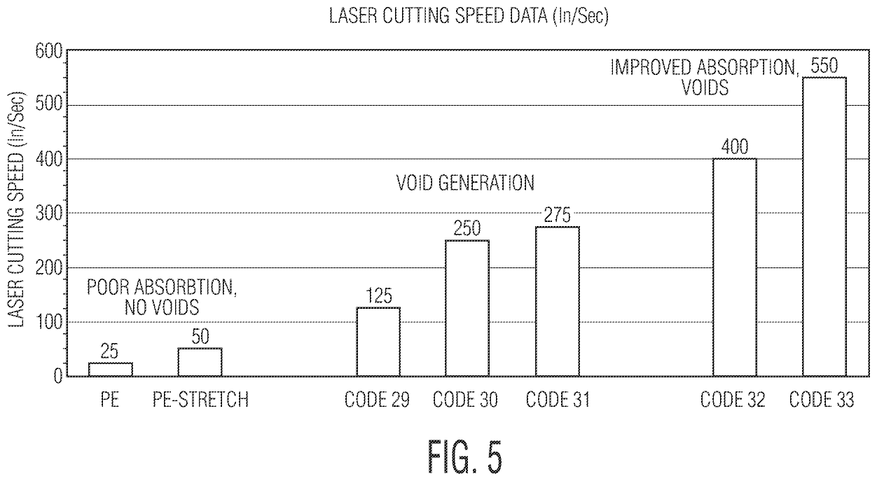

The cutting speed results for Table 5 are also depicted in FIG. 5. As shown in Table 5 and in FIG. 5, control code nos. PE and PE-Stretch demonstrate poor cutting speeds of 25 in/sec and 50 in/sec, respectively. Code nos. 29-31 included the void generating particle of calcium carbonate (CaCO₃) that provided 50% by total weight of the film 12 and differed from one another based on the amount of stretching between codes. Code nos. 29-31 demonstrated enhanced cutting speeds of 125 in/sec, 250 in/sec, and 275 in/sec, respectively, thus providing that increased stretching of the film 12 including the void generating particles of calcium carbonate were believed to provide increased void area to provide faster cutting speeds. Code nos. 32 and 33 included particles of barium sulfate (BaSO₄) at 30% by total weight of the film 12 as well as barium phosphate (BaPO₄) at 10% by total weight of the film 12. Code no. 32 was not stretched, but code no. 33 was stretched at 350%. With the particles employed in code no. 33, it is believed that the particles can provide the functionality of absorbent filler particles to provide enhanced absorption of the IR laser providing enhanced heat as well as void generating particles to provide voids in the stretch film (code no. 33) to provide the improved cutting speeds in excess of 500 in/sec.

It was also observed that if the void volume and void size generated from the absorbent filler particles and solid state stretching are not in the optimum range, their effect on the laser cutting speed becomes insignificant, as illustrated in Table 5 and FIG. 5. For code nos. 32 and 33, the BaSO₄ particles were measured to be less than 1.0 μm, on average. The small particle size for the BaSO₄ particles resulted in poor void formation and void volume percentages, and thus, not as high of cutting speeds as could be realized with particles that could provide enhanced void sizes and percentages.

It is believed that filler particle size, shape, stretching ratio, amount of loading, chemical composition and/or compatibility to the resin are factors that can play a role in the void formation. In additional experimental film code nos. 34 and 35 shown below in Table 6, when the particle size is less than 1 μm, as in the case with BaSO₄, only very small voids (0.6 μm) and void volume percentages (2%) are created after the solid state stretching. As documented in Table 6, particles of this size resulted in no improvement in laser cutting speeds. For example, code no. 34 involving an exemplary PE film 12 made with BaSO₄ filler particles had a cutting speed of 275 in/sec after stretching 400 percent as compared to code no. 35 that included the same amount of BaSO₄ filler particles, but was not stretched, and demonstrated a cutting speed of 350 in/sec. In this case, the slight differences in the cutting speeds are attributed to the differences in the film 12 thicknesses.

TABLE 6
Additional Code Listing for Codes 34 and 35

| | | | | Avg. | Maximum | |
|---|---|---|---|---|---|---|
| | | | | Void | Process | Thick- |
| Code | | Stretch | Void | Size | Cut Speed | ness |
| No. | Material | Ratio | Volume | (μm) | (in/sec) | (μm) |
| 34 | 60% PE, 40% | 400% | 2.0 | 0.6 | 275 | 20 |
| | BaSO₄ | | | | | |
| 35 | 60% PE, 40% | 0% | 1.0 | 0.3 | 350 | 13 |
| | BaSO₄ | | | | | |

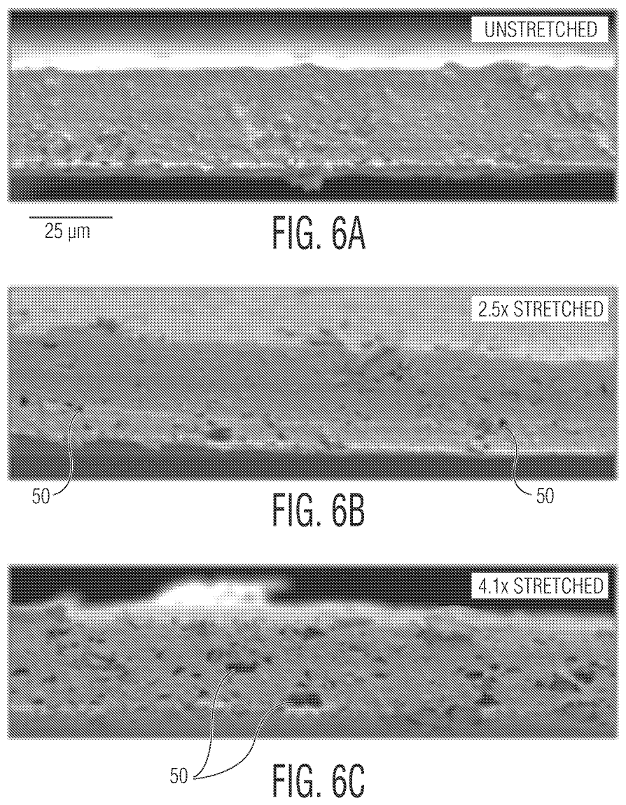

By contrast, exemplary films 12 made with CaCO₃ void generating filler particles, which have an average particle size of 2 μm, can generate significantly larger voids and void volume percentages through controllable stretching, and thus, can result in much faster laser cutting speeds. Scanning electron microscope (SEM) images were taken of experimental film layers 12 and are depicted in FIGS. 6A-6C. For example, FIG. 6A depicts the SEM image of an exemplary film 12 including void generating particles of CaCO₃, but the film 12 is in an unstretched condition. FIGS. 6B and 6C depict the same exemplary film 12 of FIG. 6A, but stretched at 250% in FIG. 6B and FIG. 6C. The voids 50 in FIG. 6C are larger and provide a greater void percentage of the film 12 as compared to the voids 50 in FIG. 6B (two voids 50 labeled in FIGS. 6B and 6C, respectively, for purposes of clarity).

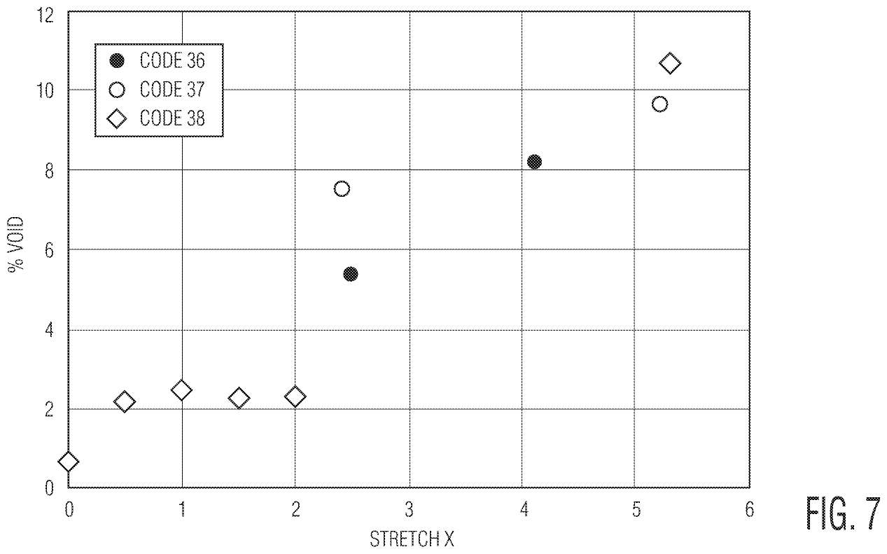

Additionally, as demonstrated in FIG. 7, increasing the amount of stretching increases the void percentage for a film 12 including void generating particles. The experimental film 12 codes from FIG. 7 are documented in Table 7 below. Code Nos. 36-38 are ABA type multilayer films that have skin layer (“A”) of composition of 70% PP, 30% BaSO₄, and the core layer (“B”) with compositions as shown in Table 7. The A/B ratio is 10/90 by weight. FIG. 7 compares the void percentage in Code Nos. 36-38 for various stretch rates of these codes.

TABLE 7
Additional Code Listing for Code Nos. 39-42

| Code No. | Sample Description |
|---|---|
| 36 | Core layer B- 59.5% LLDPE, 22.5% CaCO₃, 18% BaSO₄ |
| 37 | Core layer B- 58% LLDPE, 30% CaCO₃, 12% BaSO₄ |
| 38 | Core layer B- 56.5% LLDPE, 37.5% CaCO₃, 6% BaSO₄ |

The data in FIG. 7 shows that the void volume percentage can be from about 2% to about 15%, or from about 5% to about 15% in some preferred embodiments. It is believed that the preferable void sizes can range from about 0.6-2.0 μm. It is believed that to make void sizes and void volume percentages in the film 12 at the desirable range, the average void generating filler particle size distribution should be less than 5 μm, with a preferred particle size range of about 1-2 μm.

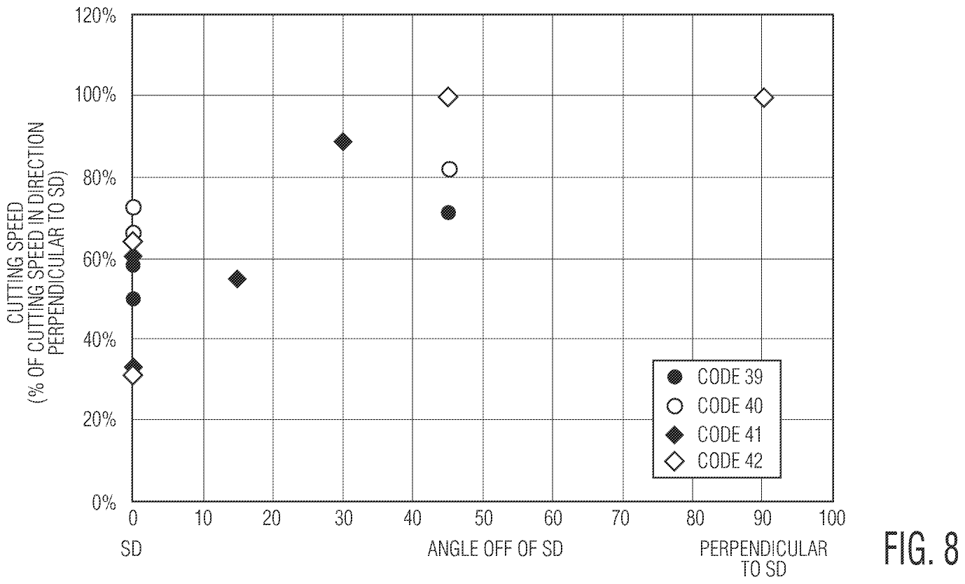

FIG. 8 demonstrates the effect of stretching ratio on the cutting speed in different directions with respect to the stretching direction (SD). FIG. 8 demonstrates cutting speed of exemplary film 12 code nos. 39-42 as a percentage of the cutting speed in the SD. For example, cutting at 0° off of the SD is cutting in the same direction as the SD, whereas cutting at 90° off of the SD is cutting in a direction that is perpendicular to the SD.

Experimental film 12 code nos. 36-39 were utilized for this testing, and their resultant description is documented in Table 8. Similar to the experimental film 12 codes nos. 36-38 of Table 7 discussed above, code nos. 39-42 are ABA type multilayer films that have skin layer (“A”) of composition of 70% PP, 30% BaSO₄, and the core layer (“B”) with compositions as shown in Table 8. The A/B ratio is 10/90 by weight.

TABLE 8
Additional Code Listing for Code Nos. 39-42

| Code No. | Sample Description | Stretch Ratio |
|---|---|---|
| 39 | Core layer B- 59.5% LLDPE, 22.5% CaCO₃, | 410% |
| | 18% BaSO₄ | |
| 40 | Core layer B- 59.5% LLDPE, 22.5% CaCO₃, | 250% |
| | 18% BaSO₄ | |
| 41 | Core layer B- 58% LLDPE, 30% CaCO₃, | 520% |
| | 12% BaSO₄ | |
| 42 | Core layer B- 58% LLDPE, 30% CaCO₃, | 240% |
| | 12% BaSO₄ | |

From FIG. 8, it appears that a higher stretching ratio results in slower cutting speeds in the SD. For example, comparing code no. 39 to code no. 40 that only differ in amount of stretch, FIG. 8 depicts that code no. 40 with lower stretch had higher relative cutting speeds in the SD. It is important to note that once the cutting angle shifts away from the SD and towards a direction that is perpendicular to the SD, such as by as little as about 10° to about 15°, the cutting speeds increase significantly.

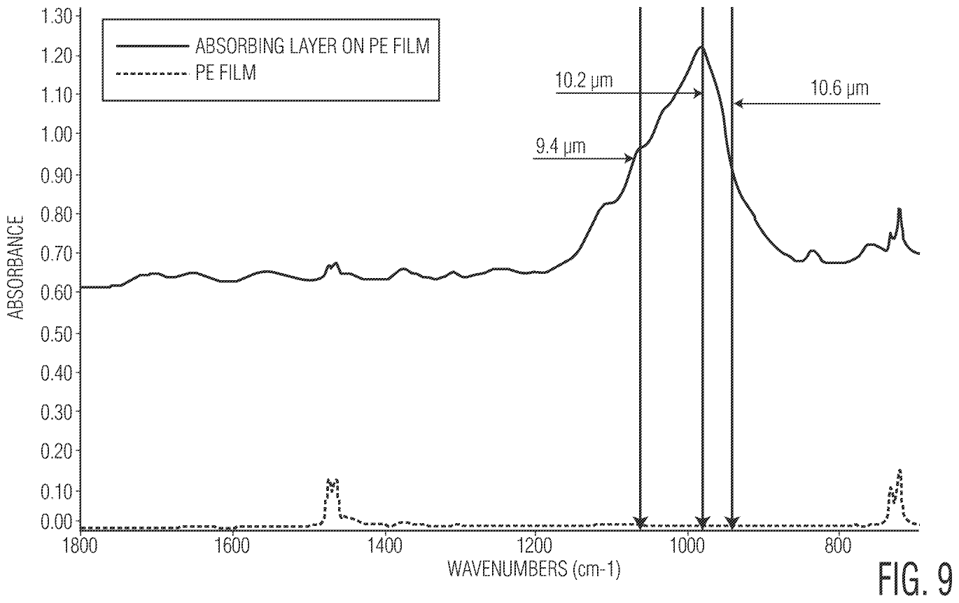

Further testing was conducted to determine if surface treatment of a highly absorbing material on a film 12, nonwoven facing material, or laminate material could provide another way to enhance cutting speed. For such testing, a coating layer of Sunchemical IR absorbent ink was provided on a PE film with add-on of between about 3 and 10 BCM (billions of cubic microns). As illustrated in FIG. 9, the coating material had high levels of IR light absorbance for wavelengths between 9.4 μm and 10.6 μm, particularly when compared to PE film alone. However, cutting speeds were not found to be affected significantly for the two samples as both the PE film with the coating and the PE film with no coating provided cutting speeds of only 25-50 in/sec using both samples. Even though the coating has a high level of absorption of the IR laser wavelength, there was not a significant increase in laser cutting speed. A cutting speed of 25-50 in/s was achieved for both the coated and uncoated films using a laser assembly at 100 W and a wavelength of 9.4 and 10.2 μm. It is believed that the absorbent coating layer does not improve the laser cutting speeds because an exemplary film 12 composed of PE has poor thermal conductivity properties and absorbent coating layer mass efficacy is low. The results of this absorbent coating testing further suggest that a laser absorbing filler particle is more effective if such particles are distributed throughout the film 12 and contribute to increased laser cutting speeds and cutting edge quality.

Thus, films 12 that include both absorbent filler particles and void generating filler particles that are stretched to provide for voids 50 lead to higher cut speeds and increased manufacturing efficiency. Another benefit that is recognized by stretching of such films 12 is an improved edge softness of the films 12, which may lead to improved comfort of products including such films 12, such as absorbent articles, as well as improved aesthetics.

### EMBODIMENTS

Embodiment 1: A film comprising: a polymer; and a plurality of particles, the plurality of particles comprising: absorbent filler particles comprising between about 1% to about 40% by weight of the film; and void generating filler particles comprising between about 10% to about 40% by weight of the film; wherein the absorbent filler particles are a different particle than the void generating particles; and a plurality of voids.

Embodiment 2: The film of embodiment 1, wherein the plurality of voids provide a void volume percentage for the film, the void volume percentage being between about 5% to about 15%.

Embodiment 3: The film of embodiment 1 or 2, wherein the plurality of voids include an average size of about 0.50 μm to about 2.50 μm.

Embodiment 4: The film of any one of the preceding embodiments, wherein the absorbent filler particles comprise between about 10% to about 25% by weight of the film and the void generating filler particles comprise between about 15% to about 35% of the film.

Embodiment 5: The film of any one of the preceding embodiments, wherein the absorbent filler particles comprise particles selected from the group consisting of: BaSO₄, BaPO₄, carbon black, kaolin, and combinations thereof.

Embodiment 6: The film of embodiment 5, wherein the absorbent filler particles comprise BaSO₄.

Embodiment 7: The film of any one of the preceding embodiments, wherein the void generating filler particles comprise particles selected from the group consisting of: CaCO₃, Alumina, silica, clay, sand, superabsorbent particles, thermal starch, cellulose fiber, and combinations thereof.

Embodiment 8: The film of embodiment 7, wherein the void generating filler particles comprise CaCO₃.

Embodiment 9: The film of any one of the preceding embodiments, wherein the polymer is a polyolefin, the polyolefin comprises polyethylene, polypropylene, or a combination thereof.

Embodiment 10: A method for cutting or perforating a web, the method comprising: providing a web including a film, the film comprising: a polymer; a plurality of particles, the plurality of particles comprising: absorbent filler particles comprising between about 1% to about 40% by weight of the film; and void generating filler particles comprising between about 10% to about 40% by weight of the film, wherein the absorbent filler particles are a different particle than the void generating particles; stretching the film to generate a plurality of voids in the film; providing a laser assembly; and directing a beam of light from the laser assembly upon the surface of the web with relative movement between the beam of light and the web to cut or perforate the web.

Embodiment 11: The method of embodiment 10, wherein stretching the film to generate a plurality of voids in the film comprises stretching the film between about 200% to about 500%.

Embodiment 12: The method of embodiment 10 or 11, wherein the plurality of voids provide a void volume percentage for the film, the void volume percentage being between about 5% to about 15%.

Embodiment 13: The method of any one of embodiments 10-12, wherein the plurality of voids include an average size of less than about 2.50 μm.

Embodiment 14: The method of any one of embodiments 10-13, wherein the plurality of voids include an average size of about 0.50 μm to about 2.50 μm.

Embodiment 15: The method of any one of embodiments 10-14, wherein the absorbent filler particles comprise between about 10% to about 25% by weight of the film and the void generating filler particles comprise between about 15% to about 35% of the film.

Embodiment 16: The method of any one of embodiments 10-15, wherein the absorbent filler particles comprise particles selected from the group consisting of: BaSO₄, BaPO₄, carbon black, kaolin, and combinations thereof.

Embodiment 17: The method of embodiment 16, wherein the absorbent filler particles comprise BaSO₄.

Embodiment 18: The method of any one of embodiments 10-17, wherein the void generating filler particles comprise particles selected from the group consisting of: CaCO₃, Alumina, silica, clay, sand, superabsorbent particles, thermal starch, cellulose fiber, and combinations thereof.

Embodiment 19: The method of embodiment 18, wherein the void generating filler particles comprise CaCO₃.

Embodiment 20: The method of any one of embodiments 10-19, wherein the beam of light from the laser assembly is a CO₂ laser comprising an excitation wavelength of between about 9.0 μm to about 11.0 μm.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A film comprising:
a polymer; and
a plurality of particles, the plurality of particles comprising:
absorbent filler particles comprising between about 1% to about 40% by weight of the film; and
void generating filler particles comprising between about 10% to about 40% by weight of the film; wherein the absorbent filler particles are a different particle than the void generating particles; and
a plurality of voids.

2. The film of claim 1, wherein the plurality of voids provide a void volume percentage for the film, the void volume percentage being between about 5% to about 15%.

3. The film of claim 2, wherein the plurality of voids include an average size of about 0.50 μm to about 2.50 μm.

4. The film of claim 1, wherein the absorbent filler particles comprise between about 10% to about 25% by weight of the film and the void generating filler particles comprise between about 15% to about 35% of the film.

5. The film of claim 1, wherein the absorbent filler particles comprise particles selected from the group consisting of: BaSO₄, BaPO₄, carbon black, kaolin, and combinations thereof.

6. The film of claim 5, wherein the absorbent filler particles comprise BaSO₄.

7. The film of claim 1, wherein the void generating filler particles comprise particles selected from the group consisting of: CaCO₃, Alumina, silica, clay, sand, superabsorbent particles, thermal starch, cellulose fiber, and combinations thereof.

8. The film of claim 7, wherein the void generating filler particles comprise CaCO₃.

9. The film of claim 1, wherein the polymer is a polyolefin, the polyolefin comprises polyethylene, polypropylene, or a combination thereof.

10. A method for cutting or perforating a web, the method comprising:
providing a web including a film, the film comprising:
a polymer;
a plurality of particles, the plurality of particles comprising:
absorbent filler particles comprising between about 1% to about 40% by weight of the film; and
void generating filler particles comprising between about 10% to about 40% by weight of the film, wherein the absorbent filler particles are a different particle than the void generating particles;
stretching the film to generate a plurality of voids in the film;
providing a laser assembly; and
directing a beam of light from the laser assembly upon the surface of the web with relative movement between the beam of light and the web to cut or perforate the web.

11. The method of claim 10, wherein stretching the film to generate a plurality of voids in the film comprises stretching the film between about 200% to about 500%.

12. The method of claim 10, wherein the plurality of voids provide a void volume percentage for the film, the void volume percentage being between about 5% to about 15%.

13. The method of claim 10, wherein the plurality of voids include an average size of less than about 2.50 μm.

14. The method of claim 10, wherein the plurality of voids include an average size of about 0.50 μm to about 2.50 μm.

15. The method of claim 10, wherein the absorbent filler particles comprise between about 10% to about 25% by weight of the film and the void generating filler particles comprise between about 15% to about 35% of the film.

16. The method of claim 10, wherein the absorbent filler particles comprise particles selected from the group consisting of: BaSO₄, BaPO₄, carbon black, kaolin, and combinations thereof.

17. The method of claim 16, wherein the absorbent filler particles comprise BaSO₄.

18. The method of claim 10, wherein the void generating filler particles comprise particles selected from the group consisting of: CaCO₃, Alumina, silica, clay, sand, superabsorbent particles, thermal starch, cellulose fiber, and combinations thereof.

19. The method of claim 18, wherein the void generating filler particles comprise CaCO₃.

20. The method of claim 10, wherein the beam of light from the laser assembly is a CO₂ laser comprising an excitation wavelength of between about 9.0 μm to about 11.0 μm.
